(19) United States
(12) Patent Application Publication
(10) Pub. No.: US 2007/0159598 A1
(43) Pub. Date: Jul. 12, 2007

(54) Pocket perimeter

(76) Inventors: Don Robert Yancey, Honolulu, HI (US); Jean-Christophe Roulet, Lignieres (CH); Christoph Kissling, Bern (CH); Urban Schnell, Munchenbuchsee (CH)

Correspondence Address: YOUNG & THOMPSON, 745 SOUTH 23RD STREET, 2ND FLOOR, ARLINGTON, VA, 22202, US

(21) Appl. No.: 11/640,174

(22) Filed: Dec. 18, 2006

Related U.S. Application Data
(60) Provisional application No. 60/754,253, filed on Dec. 29, 2005.

(51) Int. Cl. A61B 3/14

(52) U.S. Cl. 351/206

(57) ABSTRACT

A device for mapping the visual field of an eye includes a projector for creating a light beam of visible light, a first light directing element for controlling the direction of the beam, and a reflection element for reflecting the light beam through a predetermined crossing point. The elements are arranged in such a way so that the light beam can be directed to well defined sites of the retina of the eye when positioned with the center of its lens substantially in the crossing point. The projector can be made to include a focusing element and a beam cross-section forming element for creating a disk of predetermined size on the retina of the eye.

### FIELD OF INVENTION

This invention relates to a device for mapping the visual field of the eye, more particularly a perimeter.

### BACKGROUND-PRIOR ART

Scanning or mapping the visual field of the eye is useful in detecting and diagnosing various eye diseases. Such diseases detected by visual field mapping include glaucoma. Markers of glaucoma are loss of peripheral vision, increased intraocular pressure, and changes in the optic disc. If untreated, blindness ensues. If detected at an early stage, however, treatment with drugs can usually arrest development of the disease and save the patient's vision.

At present, glaucoma is diagnosed by eye specialists in a clinical setting. Instruments to map the visual field, measure intraocular pressure, and image the optic disc are bulky, complex, and expensive. These instruments require specialized skills to operate. Additionally, lengthy training is required to acquire the skills necessary to interpret results.

An example of a method to detect glaucoma is measuring the intraocular pressure (IOP) of the eye. To measure the eye's intraocular pressure a tonometer is used, which through mechanical or air-puff contact flattens an area of the cornea. The amount of flattening as indicated by the pressure required to cause the flattened area correlates to the intraocular pressure. (By measuring corneal thickness, measurements may be adjusted to increase accuracy, but measurements are aimed not at precision measurement of IOP, which varies during the day, but that IOP is within a standard range, not too high, say not below 9 mm Hg, and not too much above 20 mm Hg.)

Ideally, tonometry, measuring IOP, would be used on a routine basis to detect glaucoma in the earliest stages so that irreversible damage to the optic nerves/retina can be avoided. Then drugs could be administered to lower intraocular pressure and in most cases save the patient's vision.

But tonometry is a clinical procedure requiring specialized skills. Air puff devices to measure IOP are expensive and difficult to use. Usual mechanical tonometry similarly requires expensive equipment, although a simple version is low cost but requires even more skill to use. Mechanical tonometry can also scratch the cornea of the eye thereby risking infection and vision impairment.

In most of the world, including the advanced industrialized countries, specialized skill is not always available to detect incipient glaucoma. So the patient may not see a doctor until symptoms such as loss of peripheral vision (“tunnel vision”) become apparent to the patient. But at that point, noticeable loss of vision, the patient has already suffered irreversible damage to the retina.

According to the Merck Manual (Fourteenth Edition) measurement of intraocular pressure and mapping the visual fields should be performed semi-annually as indicated. But screening to detect incipient glaucoma and determine who should be checked semi-annually is not a routine matter: such tests are complex and costly. Only a small percentage of the world's populations is screened for glaucoma. Yet, in persons over the age of 40, glaucoma is a common and serious eye disease.

Clearly needed is a non-clinical, easy-to-use device for detecting glaucoma. This device must not only be easy to use (and low cost) but preferably non-contact. Non-contact means safe to use even by minimally skilled personnel. Ideally the device would measure IOP in a low-cost, safe, and effective manner. One type of devices for mapping the visual field in common use are the Humphrey or Octopus instruments. They project discs of varying brightness, size, or color, onto an evenly illuminated hemisphere for detecting and measuring contrast sensitivities.

### SUMMARY OF THE INVENTION

The present invention's objective is to provide a device for detecting changes in the visual field, e.g., scotomas characteristic of glaucoma.

Another object consists in proposing a device capable to scan the visual field and to perform at least a measurement of the refraction of an eye.

Accordingly, a device according to the invention produces a light disk on the retina. Preferably, the light spot is created by deflecting a corresponding beam into the eye by a concave mirror.

More preferably, the light beam is created by a beam projector and the shape of the concave mirror is such that the light disk may be produced on any point of the retina beginning from the very center to the periphery of the retina without the beam projector needing to interfere with the line of sight of the eye.

According to the other object, the concave mirror is transparent at least for light used for refractometry other than the light of the beam in the area where the line of sight crosses the mirror, so that the said light is able to pass the mirror.

My invention is preferably intended to detect and map scotomas associated with glaucoma. (That glaucoma is not easily detected by a patient or even readily apparent to patients with glaucoma, is that about 40% of the retina is affected before the patient realizes he/she has a problem.)

According to another aspect, my invention may be incorporated in an ultra-compact handheld autorefractor, either monocular or binocular. Incorporation of an autorefractor provides unique capabilities for my apparatus and methods for mapping the eye's visual field in the restricted sense of detecting and mapping scotomas associated with glaucoma.

According to a further aspect, the present invention in its preferred embodiment provides entirely objective and automatic screening of the eye to detect and assist in the diagnosis of glaucoma. Objective and automatic operation is made possible by using a CCD and microprocessor to observe, record, and analyze responses of the pupil to light stimuli projected onto various areas of the retina of the eye.

More preferably, this invention comprises an assembly consisting of a light source such as an LED and the light source is equipped with an adjustable aperture and lens so as to focus and project illuminated discs of various sizes and intensities onto the retina via a movable mirror or beam-splitter placed close to the eye, so that the illuminated discs can be projected onto any area of the retina, centrally or peripherally, by rotating the assembly and adjusting the position of the movable mirror, which is accomplished by small actuators or tiny motors such as manufactured by Sanyo. In conjunction with an integral autorefractor, position and refraction of the eye can be determined so that the subassembly LED/iris/lens can be adjusted to ensure that desired disc size and intensity is clearly focused and projected onto the targeted area of the retina.

A micro-dot hemisphere edge-lighted provides even background illumination while allowing projection of light rays (stimuli) onto the retina. The ring assembly containing LED projector can be moved in x-axis and y-axis planes to center eye pupil.

Another embodiment comprises an assembly of multiple fixed mirrors with corresponding multiple movable subassemblies comprising an LED, iris, and lens system, the subassemblies being adjustable by electromagnetic coils, and the assembly being rotated in steps by an actuator or step motor so that along with the adjustable sub-assembly illuminated discs of light can be projected onto areas of the central and peripheral retina.

Still another embodiment comprises multiple mirrors fixed in position along with corresponding multiple subassemblies of LED, iris, and lens, positions of such subassemblies being adjustable.

Still another embodiment comprises one or more tiltable light projectors, which can be moved by means of know mechanisms toward the center of eye (center of ring) so as to enable continued projection of light ray through center of pupil. Measuring z-axis distance (vertex distance) ensures that projection angles and location of projector unit from ring center continue to project light ray stimuli through center of pupil. An integral autorefractor with one or more microprocessors provides essential support such as monitoring eye center, CCD imaging of pupil, and recording/analysis of pupil size. Based on statistical analysis of initial pre-screening eye responses, microprocessors control and decide a further sequence of stimuli to eye so as to make screening efficient and rapid.

### BRIEF DESCRIPTION OF THE DRAWINGS

The invention will be further explained by way of exemplary embodiments with reference to the Drawing:

FIG. 1 Schematical side view of a embodiment of the invention;

FIG. 2 As FIG. 1, front view;

FIGS. 3-4 Second embodiment of the invention with sets of fixed mirrors turned in 20 degree steps;

FIGS. 5-6 A third embodiment of sets of fixed mirrors with fixed positions for corresponding six sets of LED/lenses;

FIG. 7 A further embodiment with movable light projectors;

FIG. 8 A fifth embodiment with tiltable projector mounted on rotatable ring;

FIG. 9 Schematic longitudinal section of a sixth embodiment;

FIG. 10 As FIG. 8, with the light projector more detailed and auto-fixation unit (AT unit);

FIG. 11 Autorefractor unit of sixth embodiment, schematic side view;

FIG. 12 Autotracking unit of sixth embodiment, schematic top (a) and side (b) view;

FIG. 13 Operator view on patient, right eye (a) and left eye (b) measurement.

### DETAILED DESCRIPTION OF THE INVENTION

While mapping the visual field alone is not sufficient to diagnose glaucoma (tonometry and examination of the optic disc for changes such as “cupping” must also be performed), detecting changes in the visual field is an early marker. One would think that anyone with glaucoma would readily notice a loss of peripheral vision. This is not true. As described above, loss of vision may not be noticed until relatively late in the process of the disease.

Loss of peripheral vision may not be noticed by the patient because the visual fields of the two eyes significantly overlap. Even inside a patient's field of vision scotomas (areas that do not receive or process images, that is, “blind spots”) the brain's visual processes may “fill in” and prevent the patient from being aware of the blind spots. An example of the brain filling in blind spots is the optic disc in each eye. Therefore, by detecting and mapping characteristic loss of peripheral vision, my invention aids in detecting incipient glaucoma.

Mapping the visual fields of the eye carefully and precisely can take about thirty minutes, depending on the skill of the physician or technician. Screening for peripheral vision may require only a few seconds, and if careful screening with an actual instrument designed for such testing is performed, only about 45 seconds per eye, and then a further few minutes to verify screening results are needed. That is, verify no scotomas (blind areas) characteristic of glaucoma.

Even simple screening of the eye requires a response from the patient, “Did you see a light, yes or no?” That is, the patient must press a button to indicate, yes, he/she saw a light stimulus. Moreover, the physician must look at the screening results and make a judgement as to whether there is a problem. My invention, however, uses physiological responses of the eye to make operation entirely objective and automatic.

The non-contact nature of this invention's functioning ensures a high degree of safety. Positive indications of glaucoma can be used to refer patients to eye specialists for a more detailed series of tests.

### FIRST EMBODIMENT

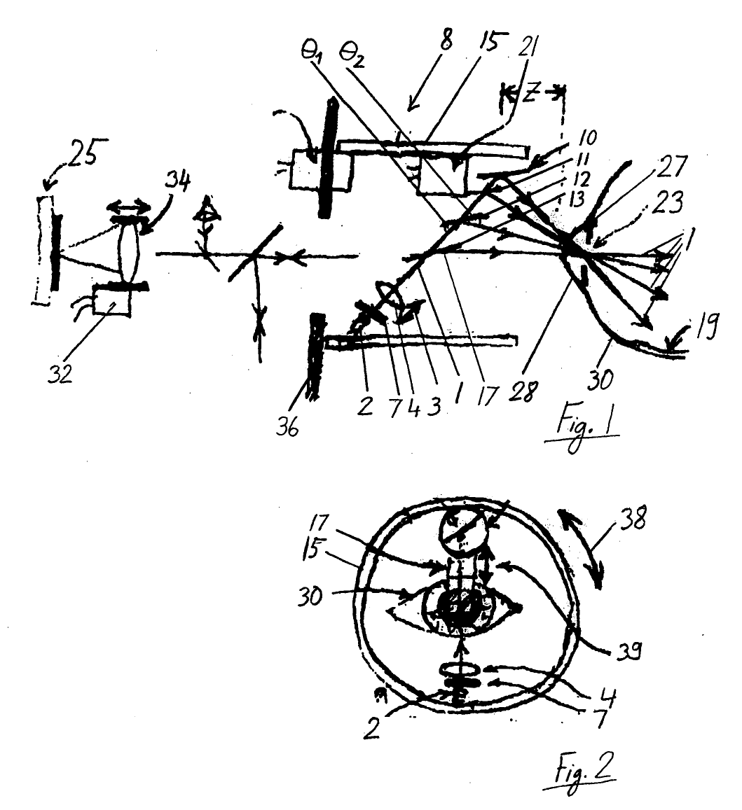

### FIG. 1

FIG. 1 shows eye 30 with light ray 1 from LED 2 with focusing lens 4 (arrow 3) and spot size adjuster iris 7, with light ray 1 reflected from movable beam-splitter (or mirror, or dichroic beam-splitter) 8. Beam-splitter 8 is shown in positions 10, 11, 12, and 13 to project spots onto periphery of retina to central area of retina. That is, the combination of a rotating cylinder or ring 15 moved by motor 16 and moving mirror 17 has the capability to project illuminated discs onto any area of the retina 19, beginning with the center up to the periphery.

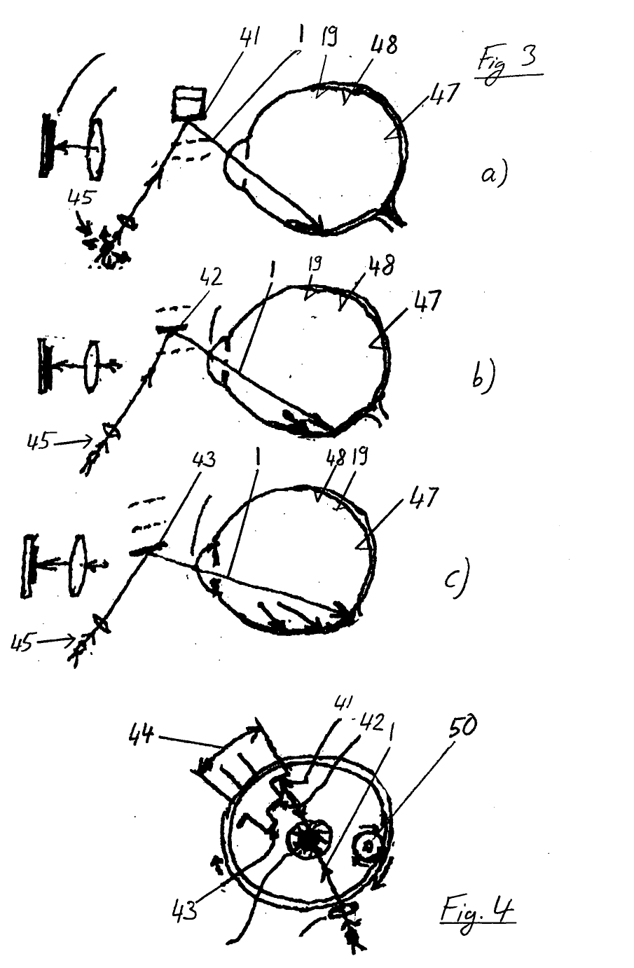

The assembly comprises a rotating ring, or rotating cylinder 15, turned by a motor 16, and with movable mirrors or beam-splitters 17 moved by motor 21, and LED/iris/lens 4 for projecting clearly defined focused discs of light of various sizes and intensities onto the central and peripheral areas of the retina 19 of the eye 30. Also shown are parts of the incorporated autorefractor including CCD 25 with adjustable lens 34 for imaging the eye 30. A microprocessor serves controlling testing sequence, recording patient responses via e.g. a pushbutton switch for the patient, and outputting the visual field map and analysis of results (not shown). FIG. 2 also shows a front view of the rotating cylinder assembly with the eye 30. Shown are motor 21 for moving the mirrors 17 and motor 16 for rotating the assembly. Shown in the rotating assembly are LED/iris/lens 2, 7, 4 for projecting a disc of light onto the retina.

The purpose of adjustable beam-splitter 8 (by action of motor 21) is to maintain a constant angle of incident and reflected light ray so that each light ray and resultant spot is projected through the pupil center 23. This is shown as Θ₁ (incident light ray angle) equal to Θ₂ (reflected light ray). Shown also is beam-splitter 8 in positions 10, 11, 12, and 13 for a multiplicity of positions wherein the light ray 1 passes each time through the pupil center 23.

Incident and reflected rays have the same angle so that the ray of light for projecting illuminated discs onto various areas of the retina always enters through the optical center of the eye.

CCD 25 locates reflected spots 27 for determining curvature of cornea 28 so that with refractive power of the eye 30, as determined by autorefractor, the iris 7 can be adjusted to project the appropriate size light spot onto the retina 19 with LED 2 being adjusted for intensity. (An ultra-sonic motor or similar device, not shown, drives the iris 7.)

The CCD 25 also monitors pupil size. Motor 32 drives imaging lens 34 so that reflected corneal spots 27, or other features of the eye 30 such as the iris, are in sharp focus so as to help determine vertex distance Z. In the case of finding the distance to a point of light, maximum intensity on a minimum number of pixels indicates the CCD lens focal point and corresponding vertex distance Z.

For a large pupil, vertex distance Z is not critical, but with a small pupil, vertex distance becomes critical. In regard to pupil size and vertex distance Z, the CCD/microprocessor monitors pupil size as well as position of the eye 30 (as determined by a corneal reflection generated by a visible fixation target, or alternately by non-visible IR) to ensure that light rays penetrate the eye through the optical center (line of sight) 23, designated here for purposes of illustration as “pupil center.”

Motor 16 fixed to instrument housing 36 turns rotating cylinder 15 so that every area of retina is mapped (FIG. 2, Arrow 38 shows assembly being rotated, Arrow 39 shows mirror 17 being moved up/down). Patient with push-button switch (not shown) indicates when a stimulus light (LED generated light spot) is detected by the patient. A processor controls testing sequences and functions to effect computerized perimetry. The resultant visual field maps assist in detecting glaucoma and related eye diseases.

FIG. 2 shows the rotating cylinder 15 as indicated by arrow 38, which is turned by motor 16 shown in cutaway. Motor 16 moves the movable mirrors 17 up/down.

### ALTERNATE EMBODIMENT

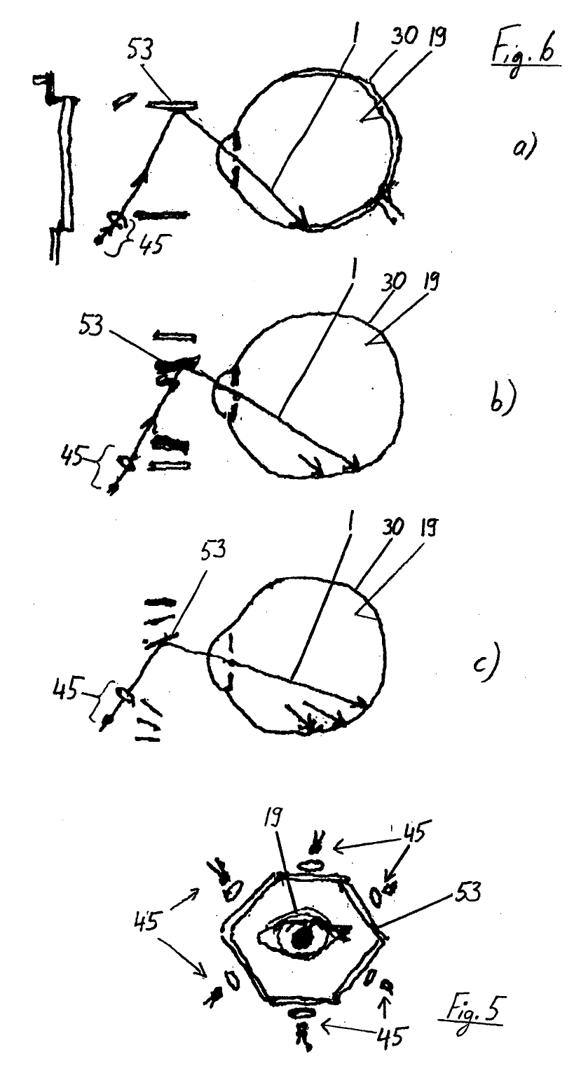

### FIGS. 3, 4

FIGS. 3, 4 shows a second embodiment of the invention with one set of six sets of fixed mirrors in a rotating ring 17. Shown are three mirrors 41, 42, 43 in 60 degrees 44 of the ring 17. Also shown associated with each of the three mirrors 41, 42, 43 is a subassembly of LED/iris/lens 45 that is fixed in position. This means that by rotating the ring 17 in steps of 20 degrees, each of the three mirrors reflects a light ray 41, 42, 43 closer or further away from the periphery of the retina 19.

In FIGS. 3, 4, moreover, the subassembly LED/iris/lens 45 sits in an adjustable electromagnetic armature so the light ray 1 is adjustable to reach all areas of the central and peripheral retina 47 resp. 48. A stepper motor 50 similar to the Sanyo T8LNP-60 is shown inside the rotating ring 17, thus indicating the miniature size of the rotating ring assembly, which could be as small or smaller than 42 mm in diameter and 14 mm in depth.

FIGS. 3, 4 shows only one of six sets 41, 42, 43 of mirrors and corresponding subassemblies of LED/iris/lens 45. This means a total of 18 distinct points of the retina 19 reached, with areas between the points reached by adjusting positions of the LED/iris/lens subassemblies 45. But it is understood that this is an example only, and the sets could be more or fewer in number, as is the case for the mirrors 41, 42, 43.

In FIG. 3, other features such as a pushbutton switch as in FIG. 1 are also applicable.

### ALTERNATE EMBODIMENT

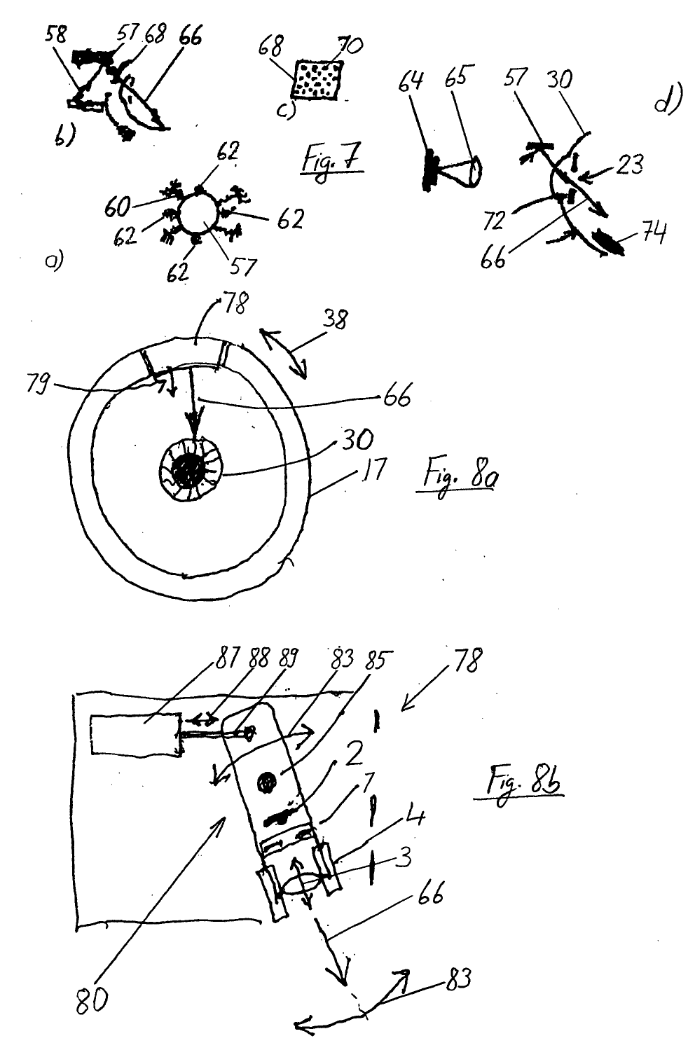

### FIGS. 5, 6

FIGS. 5, 6 shows the most simple embodiment. It comprises a fixed system of six mirrors arranged as a six-sided polygon 53 with six corresponding LEDs/lenses 45. Each LED/lens 45 projects a thin beam of light to the periphery of the retina 19.

FIGS. 5, 6 embodiment can be further developed by incorporating as shown in FIGS. 3, 4 a second and third set of polygonal mirrors. These additional mirrors with the required Θ₁=Θ₂ are added through using nested polygons or through moving the existing polygonal mirrors (separated into distinct sides) so that an iris-type mechanism with motor (not shown) rotates the mirrors into the additional positions. The additional mirror positions allow other areas of the retina 19 to be reached.

In FIGS. 5, 6, other features such as a pushbutton switch as in FIG. 1 are also applicable to the embodiment of FIGS. 5, 6.

### OTHER EMBODIMENTS

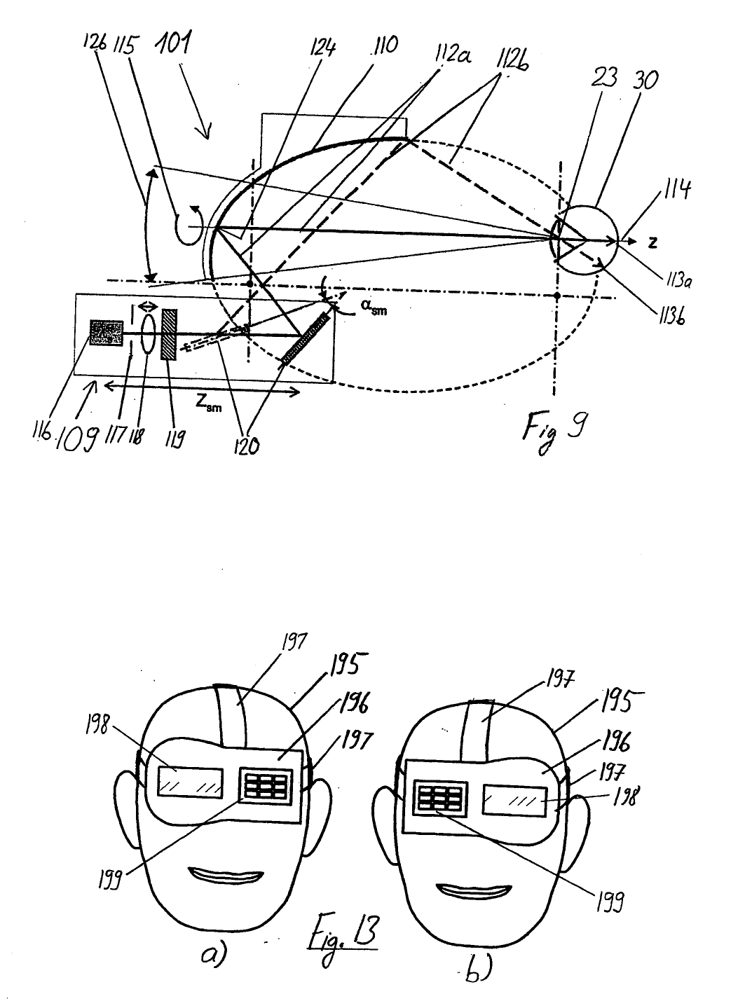

### FIGS. 7a-7d, FIGS. 8a-8b

FIG. 7a shows the ring assembly 57 comprising elements of LED projectors 58 with lenses, and motors _{———} to rotate and cause LED projectors 58 to move closer to center of eyes vision. In FIG. 7a, ring assembly 57 is represented by a circle. At 45-degree intervals, the ring assembly 57 is suspended by springs 60 (four points) and at four points in between the four suspension points are coil/magnet assemblies 62 to move the ring 57 in x-y axes so as to cause the ring 57 to be centered relative to the pupil of the eye. Note that instead of a suspension system, the ring 57 could simply be moved in x-axis and y-axis by step motors or other means.

Still referring to FIG. 7a, a fixation target light generator fixed at optical infinity in combination with a CCD 64 with lens 65 provides a corneal reflection/detection system to know center of eye 30, and a microprocessor observes and records eye's position so that the microprocessor can accordingly adjust ring's 57 x-y position so as the center the eye. Note, too, finding vertex distance z allows the ring 57 to be either moved forward or backward so projection rays 66 pass through center of pupil 23. Alternately, position of ring 57 in z-axis can be fixed and projection angles of LED 58 adjusted so as the ensure LED rays 66 pass through center 23 of pupil.

FIG. 7b shows a semi-transparent hemisphere 68 fixed in front of ring assembly 57. The hemisphere 68 is edge-lighted, preferably illuminated with low-intensity yellow light. Projected ray 58, preferably blue color and of relatively high intensity, passes through the hemisphere 68 and the center 23 of pupil as previously described.

FIG. 7c shows a detail of semi-transparent hemisphere 68. It is suitably thin so as to not introduce optical effects but allow projected ray 58 to pass through with minimal refractive effects. Note that the semi-transparent hemisphere 68 is coated with microdots 70 so that part of the projected ray 58 is blocked and the remainder of the ray passes unattenuated to the eye 30. Microdots 70 allow edge lighting of hemisphere 68 to produce a uniform lighting background. Amount of microdots 70 may vary from a few percent to fifty percent or more of hemisphere area. Microdots may be painted, etched, or applied in a number of different ways to hemisphere surface in order to produce a surface suitable for edge lighting to produce a uniform lighting background.

FIG. 7d shows apparatus and methods to automatically record and map scotomas in the retina. A lens CCD 64 images eye pupil 72 and observes changes in pupil size. A microprocessor (not shown) records sizes of eye pupil 72 in a series of pupil size measurements, say, every 10 ms to every 100 ms.

Again referring to FIG. 7d, as previously described, rays of light 66 are projected onto various areas of the retina 19, and times that rays 66 are projected, and times may be random or sequenced uniformly, are observed and recorded. The microprocessor then through a statistical analysis compares pupil sizes at times with rays projected and with rays not projected. For rays projected onto scotomas 74 there is little or no pupil change. For rays projected onto healthy retina there is change in pupil response. Knowing the area of the retina that each ray was projected for pupil response and no pupil response allows detection of scotomas 74 including scotomas 74 characteristic of glaucoma.

Still referring to FIG. 7d, note that software can perform a quick screening to detect any large scotomas 74, and then the software can allow re-examination of areas of interest, that is, areas indicating potential scotomas. Note, too, that in my invention, the intensity of projected rays as well as size of disc of projected rays onto the retina and background illumination levels are all adjustable so that subtle changes in retinal fields are more likely to be detected and mapped. Detecting subtle changes is significant in detecting and diagnosing incipient glaucoma.

FIG. 8a shows a ring assembly 17 similar to previous descriptions. This ring assembly 17 has been automatically centered in the ring 17. One subassembly 78 of actuator (micro step motor or similar device) for a pivoting light ray projector 80 is shown. As indicated in FIG. 8a this subassembly 78 can be moved toward the center of the eye 30 (arrow 79), moving in x-axis or y-axis, so as to access less peripheral areas of the retina, and more specifically for locating the eye's blind spot, a reference point for locating and mapping scotomas. Ring's rotary motion is indicated by a double-headed arrow 38. The ray projector is tiltable (arrow 83) around pin 85 by actuator 87. The actuator 87 imparts an essentially linear movement (arrow 88) on rod 89 attached to the back end of projector 80. (Actuators moving the ring toward eye center and causing rotation of ring are not shown.)

FIG. 8b shows details of subassembly 78 for rotating light ray projector 80. As light source, an LED 2 is provided. Significant is that diameter of LED light ray (and subsequent size of stimulus light disc on retina) is selectable by a variable size iris 7. Focus of disc on retina can be achieved by adjusting focal length of LED lens 4. To properly adjust focal length of LED lens 4 means knowing distance of LED from eye 30 and knowing eye's refractive measurement.

Again referring to FIG. 8b, a micro step motor or similar micro actuator causes light ray subassembly to rotate around a pin so the light ray can be projected at selected angles to reach different areas of the retina. Arrow 79 shows that the subassembly can be moved toward center of ring (eye 30) in x- or y-directions so light ray 66 continues to pass through eye's center 23 with different angles of light projection. Note that area above pin 85 to which actuator 87 is attached is not necessary, and the actuator 87 could be attached to an area of projector 80 below the pin 85 so as to make the subassembly 78 more compact.

### SIXTH EMBODIMENT

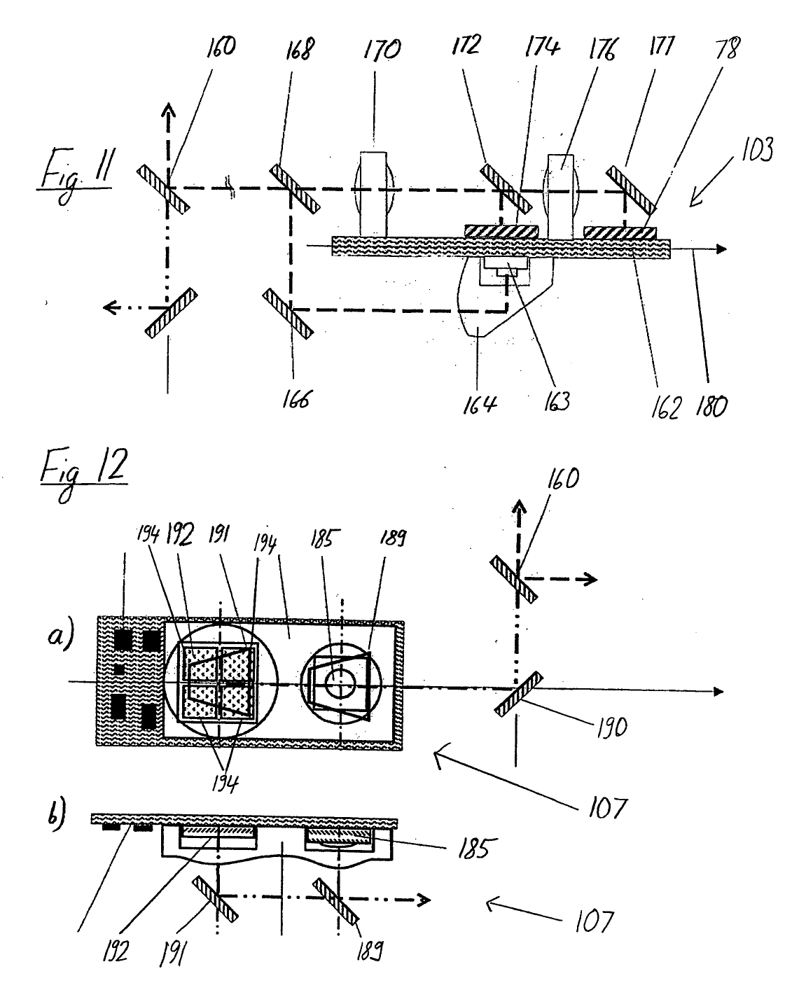

FIGS. 9-12 illustrate a sixth embodiment comprising inter alia a visual field mapping (VM) unit 101, an autorefractor unit (AR) 103 and a target fixation (TF) _{———} and auto-tracking (AT) unit 107.

The VM unit 101 comprises a light beam projector unit 109 and concave reflector 110. As in the embodiments explained above, the projector 109 generates a beam of light 112 (a, b indicating to exemplary paths of beam 112), which is directed to reflector 110. Reflector 110 deflects the beam 112 into eye 30. More specifically, the beam 112 has to pass as exactly as possible the center 23 of the lens of the eye 30 so that the site 113 (a, b corresponding to light path 112a resp 112b) where the beam hits the retina is known. If the retina is light sensitive at this site, the patient sees a light disk and confirms by pressing a button or by answering. As mentioned, with an additional unit for observing the iris of eye 30, an automatic detection if the light beam hits a light sensitive site of the retina is achieved: If the beam 112 is switched on and is seen, the iris will adapt to the increased brightness by closing, resp. it will open slightly if the beam 112 is switched off or the beam enters a non-sensitive area and vice versa.

The whole VM unit 101 is rotatable around the z-axis 114 (arrow 115), which is identical with the line of sight of eye 30 if the device is perfectly adjusted. From the foregoing it is evident that the VM unit 101 has to centered with respect to the eye 30, resp. the eye has to kept fixed to the axis of rotation of the VM unit 101 as best as possible. However, a small deviation of the lens of sight of the eye 30 and the thereby created displacement of the actually illuminated site of the retina may be numerically corrected.

The projector 109 comprises a source 116 of visible light of wavelength λₛ (e.g. a LED), an iris 117 for adjusting the width of beam 112, a lens 118 for adjusting the beam to the refraction properties of the eye 30 (the beam when hitting the retina should create a disk of constant proportions), a filter 119 for further reducing the spectrum of the light beam 112, and a movable scanning mirror 120. The lens 118 is movable as indicated by arrow 122.

By the movement of the mirror 120, the light beam 1129 can be directed to different sites on concave reflector 110. One extreme point is point 124 from which the beam is reflected exactly along the line of sight of the eye 30, i.e. the beam hits the retina in its center. The other extreme location on reflector 110 has to be a point from which the beam is directed to a peripheral area of the retina. The distance of this area from the center of the retina is not as well defined. Yet, preferably, it shall at least correspond to the most peripheral zone of the retina which should still be sensitive to light for an eye having a normal or larger area of sight.

Generally, however, both extreme positions may be chosen according to special conditions. Hence, it may be possible to pass the center of the retina or to choose other values for the peripheral boundary.

The shape of the concave reflector 110 is calculated such that the beam 112 can be directed into eye 30 as explained above. Accordingly, though apparently about elliptic or parabolic, the shape is neither of both. A reflector of such a peculiar, numerically or analytically determined shape, can be manufactured nowadays with acceptable efforts. Still to be mentioned that is particularly necessary to have the reflector 110 reflect light impinging from a direction significantly different from the z-axis into the z-axis. This allows to keep the projector 109 out of the line of sight of the eye and of the observation lines of other units like autorefractor, TF, AT unit. Furthermore, a specifically adapted shape of reflectors 110 allows as well an optimisation in order to simplify the movement of scanning mirror 120 and/or the relationship between the position of the scanning mirror 120 and the coordinates of the illuminated site of the retina of eye 30, etc.

The reflector 110 is further provided with a surface reflective in the extreme only to wavelength λₛ of beam 112. However, the reflector 110 is transparent in the area 126 about symmetrical to the z-axis, so that it is possible to examine and observe eye 30 through the reflector 110.

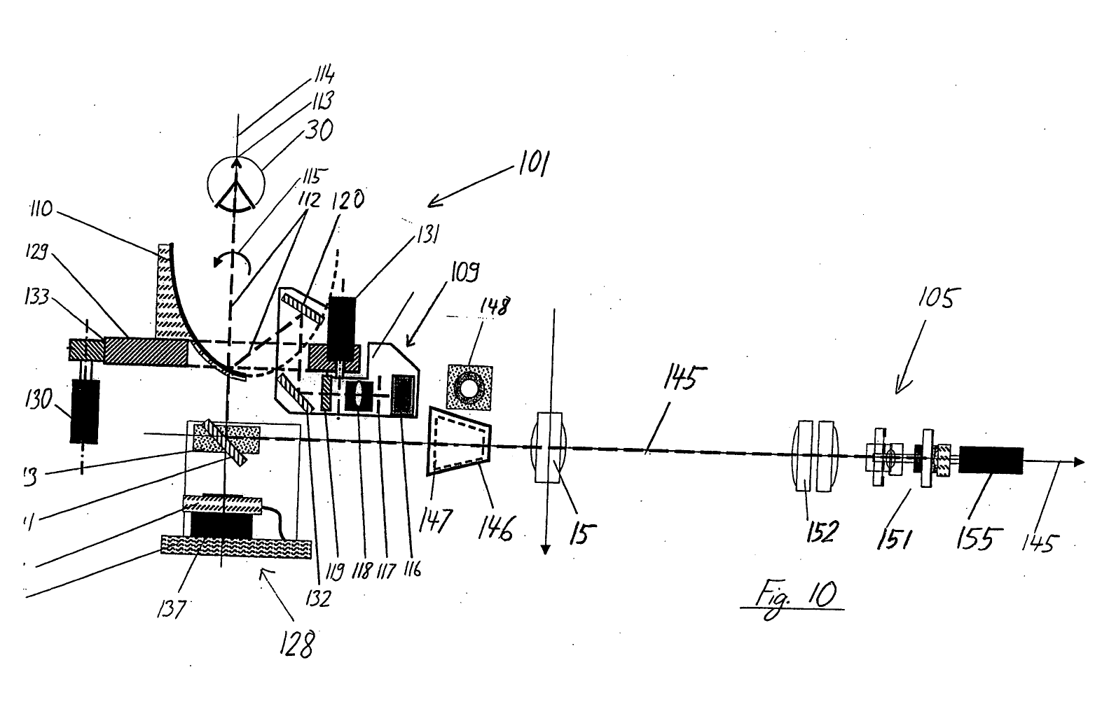

FIG. 10 shows additionally a vision unit 128, the TF unit 105 and part of AR and AT unit. Actuator 130 serves to rotate the VM unit 101 on the z-axis 114. The angular actuator 130 is coupled to the carrier 129 by e.g. toothed-wheel gearing 133. Actuator 131 moves the scanning mirrors 120. In comparison with FIG. 7 the beam generator (light source 116 up to filter 119) produce a beam which is deflected by an auxiliary mirror 132. This layout yields a more compact unit.

Still to mention that mirror 110 is only an angular segment, i.e. has the general shape of a two-dimensionally bent strip. Depending on the shape of the impinging beam, it may also be possible to use a one-dimensionally bent strip.

Hence, by rotating the whole VM unit 101 over at least 360°, the angular position of the illuminated spot on the retina of eye 30 is determined, and by moving the scanning mirror 120, the radial position, i.e. the distance to the center of the retina.

The vision unit 128 mainly serves to adjust the device as exactly as possible centered before an eye 30. It comprises a system holder 135 (possibly a PCB), on which a micro-camera 137 provided with an illumination ring 139 is mounted. The line of sight of the vision system 128, i.e. micro-camera 137, extends through a splitting mirror 141 and as explained above, through the concave reflector 110. Splitting mirror 141 is partly (e.g. 50%) transparent to visual light and reflective to IR light used by inter alia AR, AT, and TF unit. It is movable by the auto-tracking x-axis actuator 143 in the x-axis 145 vertical to the z-axis 114.

The light coming from the eye 30 and deflected by mirror 141 is deflected by mirror 146 downwards into the y-axis vertical to x-axis 145 and z-axis 114. Mirror 146 is movable by auto-tracking y-actuator 148 in direction of the y-axis. The auto-tracking actuators 143 and 148 are of a type allowing rapid movements. Preferably they are of a type comprising a movable element kept in equilibrium between two solenoids having opposedly to each other a repellent effect on the moving element.

A second splitting mirror 147 separates visual light created by the TF unit from the generally invisible IR light of AR and AT unit. The TF unit 105 comprises a target projector 151, a Badal optic 152 and a TF relay lens 153. Depending on the results of refractometry (cf. below), the target projector 151 is moved in x-direction in order to keep the target at virtual infinity. Additionally, by moving the target testwise to a distance virtually farer away, e.g. corresponding to a ¼ more hyperopic eye than measured, accommodation of the eye to infinity can be checked. If refractometric measurement yields other values thereafter, the eye has accommodated to the new distance. The process is repeated until constant refractometric values are observed meaning an as perfect as possible accommodation of the eye to infinity. TF unit is moved by the TF actuator 155.

In the light path after splitting mirror 147 follows a further splitting dichroic mirror 160 which separates the light of the AR unit 103, e.g. IR of a first, suitable wavelength, from the IR of another wavelength used by the AT unit 107.

The AR unit 103 comprises a double-sided PCB 162. On its lower side, a light source 163 is arranged. Its light is deflected and bundled by an optical element 164 toward mirror 166. Mirror 166 redirects the light to polarizing beam-splitter 168, where the light is again deflected to dichroic mirror 160.

The AR light, when reflected by the retina of eye 30 or other reflective elements, travels the same way back up to polarizing beam-splitter 168. Hence, only light whose polarization has changed or is lost which is the case with light being reflected by the retina which acts as a secondary light source, may pass beam-splitter 168. Light reflected by other elements mostly retains its polarisation and does not pass.

After beam-splitter, the light passes a refractor lens 170 and is splitted by semitransparent mirror 172. A part is deflected to near CCD sensor 174, the other part passes relay lens 176, is deflected by mirror 177 and hits far CCD sensor 178. The signals delivered by the two sensors 174, 178 are evaluated by a processor (not shown) and used to determine refraction properties of eye 30 and to adapt to the properties of the eye the system by:
moving the AR PCB 162 along axis 180;
moving the TF unit by means of actuator 155; and
moving lens 153 in the light projector 151.

The remaining part is the auto-target AT unit 107. It comprises an AT light source 185, e.g. an LED emitting IR light of a frequency different from light source 163 of the autorefractor unit. Its light is deflected by partial (e.g. semi-transparent mirror 189 and by mirror 190. Then it passes mirror 160 which is substantially transparent for this light. On return, the light follows the same path as the emitted light, and a part passes mirror 189, is deflected by mirror 191 and hits AT sensor 192. The sensor 192 comprises four sensor elements 194 having each a light sensitive surface which does not need to be further subdivided. A correct, i.e. centered adjustment of the device produces substantially the same illumination of all sensor elements 194, and a misalignment, e.g. by a rapid movement of the eye, produces differing illumination, hence signal variations. These variations used to energize actuators 143 and 148 to recenter the light paths of TF, AR, and AT unit 105, 103, and 107 respectively. Regarding the VM unit 101, a movement of the eye produces another illuminated site on the retina which is corrected numerically.

FIG. 13 shows a patient 195 with the visual field mapping device 196 arranged before his eyes. The correct position is secured by a strap or belt 197 tightened to the head on patient 195, preferably together with a headrest or the like for securing a fixed distance of the device 196 from the eyes of the patient. The correct position is observed by the image on the screen 198 furnished by the vision unit 28. The keyboard 199 allows controlling the device by the operator.

The device further comprises an acceleration or gravity sensor. As FIG. 13 shows, the device is turned upside down if used for the other eye. The gravity sensor allows to detect automatically if a left or right eye is measured, and besides adapts the image on screen 198 and rearranges the functions of the keys of keyboard 199 accordingly.

Advantages

An advantage of the present invention is its ultra-compact size, that is, reducing current visual field instruments from bulky table-top apparatus to an ultra-compact device.

A further advantage of the present invention is that it is relatively low-cost compared to present expensive instruments, so that affordability makes visual field mapping apparatus available and accessible to large populations in vast geographical regions.

Still another advantage is that this ultra-compact and battery-powered invention allows the invention to be carried and used almost anywhere.

Another advantage is that the easy-to-use handheld size enables non-specialists such as internists, family physicians, and paramedicals to perform vision testing for patients with hitherto limited or no access to eye care.

Other advantages not detailed here will become apparent as the present invention is more fully described in the ensuing pages.

Operation

As indicated in the above descriptions and in the Drawings and to wit: The physician or other practitioner asks the patient to look into the invention, and look at the fixation target. The patient is asked to press a pushbutton, held in the patient's hand, each time the patient perceives a flash of light, all the while looking forward at the fixation target. Sequence of targets and recording and analysis as well as print-out or other display of the data is thenceforth automatic.

Operation of my invention to detect glaucoma not requiring patient response is entirely automatic. Pupil response is recorded and compared to light ray stimuli objectively and automatically to detect and map scotomas characteristic of glaucoma.

In a regular patent application, operation will be described in detail using specifications described in these pages.

Conclusions, Ramifications, and Scope

Conventional visual field mapping instruments are difficult to use and accessible to only a very small percentage of the world's population, yet in most of the world eye diseases such as glaucoma are common but undetected and undiagnosed until the patient has irreversible damage to the eye, usually leading to blindness.

My invention, ultra-compact apparatus and methods for detecting and mapping scotomas characteristic of incipient glaucoma, has the advantages of small handheld size, complete portability, easy use, and low cost. Moreover, operation can be made entirely objective and automatic, not requiring patient response.

In conclusion, this invention helps make eye care accessible and affordable to large populations in vast geographical regions. Detection and diagnosis of an eye disease such as glaucoma can then be treated with drugs to help arrest the disease and prevent blindness.

From the description of embodiment given above, numerous variations and alternations are accessible to the one skilled in the art without leaving the scope of protection which is defined solely by the claims. One modifications may be to design the convex reflector 110 as a sequence of plane mirrors. This allows to only stepwise move the beam in radial direction, i.e. only rings of discrete distances from the center of the retina can be scanned. However, construction of this mirror may be less complicate.

As is mentioned in the introduction, it is further conceivable to have the device contain two mapping units so that two eyes may be mapped without moving and readjusting the device. Evidently, the distance between the two mapping units has to be adjustable in order to adapt the device to the interpupillary distances differing between persons.

### Glossary

AR auto-refractor
AT auto-tracking
IR Infra-red
PCB printed circuit board
TF target fixation
VM visual field mapping

1. A device for mapping the visual field of an eye comprising
a projector means for creating a light beam of visible light,
a first light directing means for controlling the direction of of the beam, and
a reflection means for reflecting the light beam through a predetermined crossing point,
so that the light beam is directable to well defined sites of the retina of the eye when positioned with the center of its lense substantially in the crossingpoint.

2. The device of claim 1, wherein the projector means comprises a focussing means and a beam cross-section forming means for creating a disk of predetermind size on the retina of the eye.

3. The device of claim 2, wherein the device comprises
a refractometer means for determining the refraction properties of the eye and
controller means operably connected to the refractometer means in order to receive the refractometric values from the refraction means and connected to the beam focussing means and the beam cross-section shaping means,
so that the properties of the beam are adjustable depending on the refractometric properties of the eye.

4. The device of claim 1, wherein the first directing means comprises a reflection means having at least two reflection zones and second directing means for directing the light beam to the reflection zones, the reflection zones being arranged to reflect the beam to the crossing point.

5. The device of claim 4, wherein a first one of the reflection zones reflects the light beam substantially along the vision line to the crossing point.

6. The device of claim 4, wherein a second one of the reflection zones reflects the light beam with an angle of at least 45° (???) with respect to the vision line through the crossing point.

7. The device of claim 4, wherein the reflection means is substantially a concave mirror.

8. The device of claim 4, wherein the reflection means extends over the vision line.

9. The device of claim 8, wherein the device comprises at least one means for examining an eye located at the crossing point by means of radiation, the radiation being different from the light produced by the projector and the reflection means being sufficiently transmissive to the radiation to permit the examination by the examination means.

10. The device of claim 9, wherein the radiation is light of a wavelangth different from the light of the projector.

11. The device of claim 1, wherein the projector means and the first directing means are arranged to be rotatable around the vision line and coupled to be synchronously rotated, so that the light beam is angularly movable.

13. The device of claim 1, wherein the first directing means comprises a reflecting means movable radially to the vision line and tiltable around an axis substantially vertical to the vision line and the radial movement direction.

14. The device of claim 1, wherein an eye observation means for observing the reaction of the iris on light, so that a movement of the iris in reaction of the light beam hitting a light sensible area of the retina of the eye is detectable by the eye observation means.

15. The device of claim 1, wherein a gravity sensor is provided for sensing a component of the direction of the weight force, the component being substantially vertical to the vision line, and a controller is present in operable connection to the sensor so that the device can be reconfigured by the controller according to at least two substantially opposite orientations with respect to the weight force, the two orientations being vertical to the vision line.
